(19) United States
(12) Patent Application Publication
(10) Pub. No.: US 2020/0085703 A1
(43) Pub. Date: Mar. 19, 2020

(54) HAIR CARE PRODUCTS WITH SPECIAL RENEWABLE RAW MATERIALS

(71) Applicant: Henkel AG & Co. KGaA, Duesseldorf (DE)

(72) Inventors: Sylvia Kerl, Boenningstedt (DE); Tina Ahnfeldt, Hamburg (DE)

(73) Assignee: Henkel AG & Co. KGaA, Duesseldorf (DE)

(21) Appl. No.: 16/553,481

(22) Filed: Aug. 28, 2019

(30) Foreign Application Priority Data
Sep. 17, 2018 (DE) 10 2018 215 749.9

(51) Int. Cl. A61K 8/06; A61K 8/31; A61K 8/89; A61K 8/37; A61K 8/891; A61K 8/35; A61K 8/41; A61K 8/65; A61K 8/42; A61K 8/892; A61Q 5/12

(52) U.S. Cl. CPC A61K 8/062; A61K 8/31; A61K 8/89; A61K 8/375; A61K 8/891; A61Q 5/12; A61K 8/416; A61K 8/65; A61K 8/42; A61K 8/892; A61K 8/35

(57) ABSTRACT

Hair treatment products are provided in the form of two-phase systems with two separate, visually discernible continuous phases with a common vertical phase boundary, containing one water phase (I) and one oil phase (II),
wherein the oil phase (II) contains at least about 10% by weight of at least one linear C₉-C₁₅ alkane a) relative to the total weight thereof, and
wherein the water phase (I) contains at least about 75% by weight water relative to the total weight thereof. The hair treatment products are particularly suitable for improving
the feel, particularly of dry hair
the styling properties of hair
the smoothness and softness of hair (anti-frizz).

### CROSS-REFERENCE TO RELATED APPLICATION

This application claims priority to German Patent Application No. 10 2018 215 749.9, filed Sep. 17, 2018, which is incorporated herein by reference in its entirety.

### TECHNICAL FIELD

The application describes hair treatment products in the form of a two-phase system with one oil phase and one water phase in the form of two separate, optically discernible, continuous phases with a common, vertical phase boundary, wherein the oil phase includes a large fraction of at least one linear C₉-C₁₅ alkane, and the water phase includes a large fraction of water.

The application further describes a method for conditioning hair using the hair treatment products and the use of the hair treatment products for improving specific care properties of hair.

Finally, the application also describes the use of one or more linear C₉-C₁₅ alkane(s) in cosmetic two-phase systems with two separate, optically discernible, continuous phases with a common, vertical phase boundary, for improving the speed of separation and the complete separation of the phases after they have been mixed.

### BACKGROUND

Two-phase hair conditioning products have been known from the related art for a long time and are valued highly by consumers for their attractive appearance.

They are typically leave-on products, which are preferably applied to the hair (preferably sprayed) immediately after the two phases have been mixed briefly and groom the hair.

During the manufacture of such products, one of the difficulties encountered is how to stabilise the two continuous phases. On the one hand, it should be possible to mix the two phases quickly and easily (by brief, vigorous shaking) before applying them to the hair, on the other hand they should separate equally quickly in the resting state and regain their visually attractive appearance (including a well-defined phase boundary).

In order to achieve a rapid, thorough mixing and separation, in the past frequent use was made of silicones, special polymers and/or polyols:

In EP 2025318, two-phase hair conditioning products are described which contain arylated silicones in the oil phase.

WO 2010/069995 describes two-phase products with a high polyol content in the water phase and a mixture of linear alkanes and silicones in the oil phase.

In EP 2444056 volatile silicones are suggested for a particularly good separation of the phases.

However, there is still a need for improvement with regard to a number of care aspects. Furthermore, from ecological viewpoints the desire exists on the part of the consumers for compositions with good care properties having as high a water content as possible.

### BRIEF SUMMARY

Hair treatment products, methods of treating hair, and use of alkanes in hair treatment products are provided. In an exemplary embodiment, a hair treatment product comprises a water phase (I) and an oil phase (II) that are separate, visually discernible, and continuos. The phases have a common vertical phase boundary. The oil phase (II) includes at least about 10% by weight of at least one linear C₉-C₁₅ alkane a), relative to a total weight of the oil phase (II). The water phase (I) includes at least about 75% by weight water, relative to a total weight of the water phase (I).

A method of treating hair is provided in another embodiment. The method includes vigorously shaking a hair treatment product, where the hair treatment product comprise a water phase (I) and an oil phase (II) that are separate, visually discernible, and continuous, and where the two phases (I) and (II) have a common vertical phase boundary. The hair treatment product is applied onto wet or dry hair. The hair treatment product is left on the hair until the next time the hair is washed.

Use of an alkane in a hair treatment product is provided in another embodiment. The use includes using one or more linear C₉-C₁₅ alkane(s) in a cosmetic, two-phase system having two separate, visually discernible continuous phases, where the two phases have a common vertical phase boundary. The use includes improving the speed of separation of the cosmetic two-phase system, and providing a complete separation of the phases after the cosmetic two-phase system has been thoroughly mixed.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The object underlying the present application was therefore to provide hair treatment products in the form of two-phase systems with good care performance which assure an optimal balance between long-lasting, sustainable care and good tolerability.

At the same time, the hair treatment products should contain as high a water content as possible with consistent care performance, and in particular should make previously damaged and/or dry hair and/or split ends still softer, smoother and silkier.

A further object included providing two-phase hair treatment products, the phases of which separated again quickly and completely in the resting state after mixing, so that no turbidities are visible at the phase boundary.

Yet another objective was to provide two-phase hair treatment products in which at least one of the phases (oil and/or water phase) is transparent.

It was found that the abovementioned requirements could be largely satisfied with a content of special linear alkanes in the oil phase and a high water content in the water phase.

Hair treatment products of such kind in the form of two-phase systems with a continuous oil phase and a continuous water phase can be mixed and separated again particularly quickly and thoroughly. They have good thermal and storage stability.

Moreover, they have a demonstrable conditioning benefit for hair that is treated with them. Particularly the feel (smoothness, softness, silkiness) and the hairdressing and styling properties particularly of previously stressed and/or damaged hair could be improved significantly. And the “anti-frizz” effect of such hair treatment products was assessed as excellent in a consumer test.

The hair treatment products as contemplated herein comprise two visibly separate phases which can be homogenised by shaking and in the resting state separate again into two separate, visibly distinct continuous phases with a shared vertical phase boundary.

Ideally, the hair treatment products as contemplated herein may be seen to have regained their original state with a vertical phase boundary within ten hours, preferably within seven hours, particularly preferably within five hours and more particularly within three hours.

For rapid mixing and separation of the two phases, it may be advantageous if the water phase (I) and the oil phase (II) have relatively low viscosity values. Within the scope of the present disclosure, “relatively low viscosity values” are understood to be preferably viscosity values up to a maximum of about 1000 mPas, more preferably about 600 mPas and particularly about 400 mPas (measured at about 20° C. with a Brookfield DV-II viscometer, spindle 2 at about 20 rpm) for both the water phase (I) and the oil phase (II).

For better discernibility of the mixing and separation of the two phases, it may further be advantageous if the water phase (I) and/or the oil phase (II) is (are) transparent.

With this condition, a sufficiently good mixture of the two phases can preferably be observed in that only a single phase is visible, which may be transparent to opaque.

Particularly preferably, at least one of the two phase (I) and (II) is transparent.

Within the scope of the present disclosure “transparency” is understood to mean that the water phase (I) and/or the oil phase (II) in the resting state preferably has (have) a NTU value (Nephelometric Turbidity Unit) not above about 100, preferably not above about 75, more preferably not above about 50 and more particularly not above about 25 (measured for example with a Turbiquant® type turbidimeter manufactured by Merck).

One or both phases (I) or (II) may be coloured with a cosmetically acceptable dye to allow improved visibility.

It is further desirable and preferable that the hair treatment products as contemplated herein are sprayable.

In sprayable form, the hair treatment products as contemplated herein are suitable for application using a pump dispenser. The advantage of such an application form is that it can be manipulated easily, cleanly and quickly, because the hair treatment products can be expelled from a suitable pump dispenser in the form of a fine spray mist and reach all areas of the hair simply by operating a pump valve.

In this way, the hands do not come into contact with the product and do not have to be cleaned after application of the product.

For such an application form, the application-ready (i.e., homogenised by shaking) hair treatment products as contemplated herein preferably have a viscosity not greater than about 1000 mPas, preferably not greater than about 800 mPas, more preferably not greater than about 600 mPas and particularly not greater than about 500 mPas (measured at about 20° C. with a Brookfield DV-II viscometer, spindle 2 at about 20 rpm).

Also preferable for such an application form is that the hair conditioning products as contemplated herein contain no propellants.

In a preferred embodiment, the hair treatment products as contemplated herein contain
the water phase (I) in a weight proportion from about 70 to about 98% by weight of the total weight of the hair treatment product, and
the oil phase (II) in a weight proportion from about 2 to about 30% by weight of the total weight of the hair treatment product.

It is further preferable within the scope of the present disclosure if the water phase (I) contains at least about 80% by weight, preferably at least about 85% by weight and particularly at least about 90% by weight water relative to its total weight.

According to a further preferred embodiment, the oil phase (II) contains at least about 15% by weight, preferably at least about 20% by weight, more preferably at least about 25% by weight and particularly at least about 30% by weight of at least one volatile linear C₉-C₁₅ alkane a) (wherein the quantities indicated relate to the total weight of the oil phase (II)).

Alkanes a) that are understood to be suitable as contemplated herein are preferably volatile linear saturated C₉-C₁₅ alkanes a) and particularly mixtures of said alkanes.

Within the scope of the present disclosure, “volatile alkanes a)” are preferably linear, saturated C₉-C₁₅ alkanes which have a boiling point in the range from about 60° to about 260° C., particularly preferably n-nonane, n-undecane, n-tridecane, n-tetradecane and/or n-pentadecane.

Particularly preferred alkanes a) for use in the hair treatment products as contemplated herein are mixtures of the aforementioned volatile linear saturated alkanes a), which contain at least about 85% by weight of a mixture of n-undecane and n-tridecane (wherein the quantities indicated relate to the total weight of the mixture of volatile linear saturated C₉-C₁₅ alkanes a)).

Such alkanes a) and mixtures of alkanes a) are available commercially, for example with the trade name Cetiol Ultimate® from BASF.

In a further preferred embodiment of the present disclosure, hair treatment products as contemplated herein contain in the oil phase (II) a mixture of volatile linear saturated C₉-C₁₅ alkanes a), preferably a mixture containing at least about 85% by weight of at least two alkanes selected from n-nonane, n-undecane, n-tridecane, n-tetradecane and n-pentadecane, and particularly a mixture containing at least about 85% by weight n-undecane and n-tridecane (wherein the quantities indicated relate to the total weight of the mixture of volatile linear saturated C₉-C₁₅ alkanes a)).

It was found that a particularly rapid and complete separation of the water phase (I) and the oil phase (II) can be achieved if the hair treatment products as contemplated herein further contain at least one silicone b), preferably at least one volatile silicone b) in the oil phase (II) in addition to the at least one alkane a). In such a case it is advantageous if the weight proportion of all volatile alkanes a) to all volatile silicones b) in the oil phase (II) is equal to from about 1:3 to about 2:1, particularly preferably from about 1:2 to about 1:1.

In a further preferred embodiment, besides at least one silicone b) the hair treatment products as contemplated herein further contain preferably at least one volatile silicone b) with a boiling point from about 60 to about 260° C. in the oil phase (II).

The at least one volatile silicone b) is used in the oil phase (II)—relative to the total weight thereof—preferably in a quantity of at least about 20% by weight, particularly preferably at least about 25% by weight and particularly about 30% by weight.

Suitable volatile silicones b) may be cyclic or linear. They are preferably chosen from compounds according to one of the following formulas (i) to (iii)

(CH₃)₃—Si—O—[(CH₃)₂Si—O]ₙ—Si(CH₃)₃ (i),

(CH₃)₃—Si—O—[(CH₃)₂Si—O-]ₙ-[(CH₃)Si(R)—O-]ₘ-Si(CH₃)₃ (ii),

cyclic compounds containing the formula unit —[(CH₃)₂—Si—O]ₒ— (iii),

(OCH₃)₃—Si—C₆H₅ (iv),

in which
n and m each stand for integers from 0 to about 8,
o stands for integers from 3 to about 7, and
n+m stands for the digits 0, 1, 2, 3, 4, 5, 6, 7 and 8, and
R stands for a C₂-C₈-alkyl group, an aryl group or a C₂-C₈-hydroxyalkyl group.

n, m each stand preferably for an integer from 0 to 5, particularly preferably for 0, 1 or 2, and R stands for a C₂-C₄ alkyl group.

Most particularly preferred volatile silicones b) for use in the hair treatment products as contemplated herein are the compounds known by the names hexamethyldisiloxane, octamethyltrisiloxane, decamethyltetrasiloxan, hexamethylcyclotrisiloxane (D3), octamethylcyclotetrasiloxane (D4), decamethylcyclopentasiloxane (D5), dodecamethylcyclohexasiloxane (D6) and/or phenyl trimethicone.

They may be used in the hair treatment products as contemplated herein individually or in combination.

For some embodiments, it may be advantageous if the volatile silicones b) are used in the hair treatment products as contemplated herein in combination with smaller quantities of less volatile or non-volatile silicones b-i).

Examples of particularly suitable silicones b-i) within this embodiment are for example
silicones modified with alkylene oxide groups, preferably with EO- and/or PO-groups, for example PEG/PPG(x/y)-dimethicone,
silicones modified with alkylene oxide groups and alkyl groups, for example R-PEG/PPG(x/y) dimethicone,
silicones modified with hydroxyl groups and/or amino groups, wherein the indices x and y each stand for the number of PEG- and PPG-groups, and R stands for an alkyl radical.

The one or more silicone(s) b-i) may be used in the oil phase (II) preferably in a quantity of from about 0-30% by weight, particularly preferably from about 3-25% by weight and particularly from about 5-20% by weight relative to the total weight thereof.

In a particularly preferred embodiment, the oil phase (II) contains—relative to the total weight thereof—
from about 10-60% by weight, preferably from about 20 to about 50% by weight of at least one linear C₉-C₁₅ alkane a),
from about 20 to about 80% by weight, preferably from about 30 to about 60% by weight of at least one volatile silicone b), and
0 to about 30% by weight preferably from about 5 to about 20% by weight of at least one silicone b-i).

Within this embodiment, it is further preferable if a mixture of volatile silicones, for example a mixture of volatile silicones known by the INCI names Cyclomethicone and Phenyl Trimethicone is used as volatile silicone b).

Within this embodiment, it is also referable if silicone emulsifiers (such as compounds known by the INCI names PEG/PPG(x/y) dimethicone and/or R-PEG/PPG(x/y) dimethicone) and/or compounds known by the INCI name Dimethiconol are used as silicone b-i).

In order to optimise the care properties of the hair treatment products as contemplated herein, it is advantageous if hair care ingredients are also added to the water phase (I). When choosing these care ingredients, it must be ensured that they do not interfere with the stability of the water phase (I) or the stability of the two-phase product.

Ideally, therefore, only care ingredients which are readily soluble in the water phase (I) and/or do not increase the viscosity of the water phase to values outside of the limits stated previously are used in the water phase (I).

Examples of suitable (care) ingredients which may be added to the water phase (I) advantageously are selected from
cationic tensides and/or
cationic polymers and/or
protein hydrolysates and/or
vitamins and/or
pH-regulating agents.

The water phase may also contain preservatives, for example benzoic acid and/or a physiologically tolerable salt of benzoic acid.

In a further preferred embodiment, the hair treatment products as contemplated herein contain from about 0.01 to about 0.5% by weight of at least one cationic tenside c)—relative to the total weight of the water phase (I)—in the water phase (I).

The cationic tensides c) are used particularly preferably in quantities from about 0.05 to about 0.4% by weight, most particularly preferably from about 0.075 to about 0.3% by weight and particularly from about 0.10 to about 0.2% by weight (relative to the total weight of the water phase (I)), as they are intended to reinforce the hair care effect but not encourage an extension of the emulsification of the oil phase (II) (or of parts of the oil phase).

Cationic tensides c) considered suitable for use in the hair treatment products as contemplated herein are understood to be preferably linear or branched, saturated or unsaturated physiologically tolerable C₁-C₂₄ alkyltri(C₁-C₄)alkylammonium salts, Di-C₁-C₂₄-alkyldi(C₁-C₄)-alkylammonium salts or tri-C₁-C₂₄-alkyl(C₁-C₄)alkylammonium salts and/or primary, secondary or tertiary amines containing at least one R—CO(NH)—(CH₂)ₙ group, in which R stands for a linear or branched, saturated or unsaturated alkyl group with from about 7 to about 21 C atoms, and n stands for an integer from 1 to 4.

Particularly preferred C₁-C₂₄-alkyltri(C₁-C₄)alkylammonium salts, di-C₁-C₂₄-alkyldi(C₁-C₄)-alkylammonium salts, and tri-C₁-C₂₄-alkyl(C₁-C₄)alkylammonium salts are, for example, halide and/or methosulfate salts, particularly chlorides, bromides and methosulfate, such as for example distearyl dimethyl ammonium chloride, lauryl trimethyl ammonium chloride, lauryl trimethyl benzylammonium chloride, tricetyl methyl ammonium chloride, cetyl trimethyl ammonium chloride, cetyl trimethyl ammonium bromide, cetyl trimethyl ammonium methosulfate, stearyl trimethyl ammonium chloride, behenyl trimethyl ammonium chloride, behenyl trimethyl ammonium bromide and behenyl trimethyl ammonium methosulfate. Most particularly preferred are cetyl trimethyl ammonium and behenyl trimethyl ammonium salts, and particularly cetyl trimethyl ammonium salts which contain a methosulfate ion and/or a chloride ion as the anion.

Particularly preferred primary, secondary or tertiary amines that contain at least one R—CO(NH)—(CH₂)ₙ group, in which R stands for a linear or branched, saturated or unsaturated alkyl group with from about 7 to about 21 C atoms, and n stands for an integer from 1 to 4, are known by the name “amidoamine”.

Suitable amidoamines may be present in the hair conditioning products both as such and in the respective form of the corresponding quaternary compound thereof (as a result of a protonation in a correspondingly acid solution).

The non-cationic amidoamines are preferred.

Particularly suitable amidoamines, which may optionally be quaternised, are for example Tego Amid® S18 (Evonik: INCI name: Stearamidopropyl Dimethylamine), Lexamine® S13 (Inolex: INCI name: Stearamidopropyl Dimethylamine), Incromine® SB (Croda: INCI name: Stearamidopropyl Dimethylamine), Witcamine® 100 (Witco, INCI name: Cocamidopropyl Dimethylamine), Incromine® BB (Croda, INCI name: Behenamidopropyl Dimethylamine), Mackine® 401 (McIntyre, INCI name: Isostearylamidopropyl Dimethylamine) and other Mackine types as well as Adogen® S18V (Witco, INCI name: Stearylamidopropyl Dimethylamine).

The following are examples of products which may be used as permanent cationic amidoamines: Rewoquat® RTM 50 (Witco Surfactants GmbH, INCI name: Ricinoleamidopropyltrimonium Methosulfate), Empigen® CSC (Albright & Wilson, INCI name: Cocamidopropyltrimonium Chloride). Swanol Lanoquat® DES-50 (Nikko, INCI name: Quaternium-33), Rewoquat® UTM 50 (Witco Surfactants GmbH, Undecyleneamidopropyltrimonium Methosulfate).

Particularly preferred is Stearamidopropyl Dimethylamine.

In a further preferred embodiment, the hair treatment products as contemplated herein contain from about 0.01 to about 0.5% by weight of at least one cationic polymer d)—relative to the total weight of the water phase (I)—in the water phase (I).

Cationic polymers d) that are suitable for use in the hair treatment products as contemplated herein are preferably understood to be cationic polymers known by the INCI name “Polyquaternium”.

Particularly suitable cationic polymers are Polyquaternium-6, Polyquaternium-7, Polyquaternium-10, Polyquaternium-11, Polyquaternium-16, Polyquaternium-22, Polyquaternium-24, Polyquaternium-28, Polyquaternium-37, Polyquaternium-67. Polyquaternium-74 and Polyquaternium-89.

Particularly suitable are cationic polymers that are derived from polymers of natural origin such as cellulose polymers, starch polymers or guar polymers.

Cationic cellulose derivatives are marketed for example by the company Amerchol with the name Polymer JR® and cationic guar polymers are marketed by Rhône-Poulenc or Hercules with the names Jaguar® or N-Hance®.

Further suitable cationic polymers of natural origin are for example cationic chitosans, which are available commercially with the names “Kytamer” or “Hydagen” for example.

Also suitable are the cationic polymers known by the INCI name “Quaternium”, such as Quaternium-8, Quaternium-14, Quaternium-15, Quaternium-18, Quaternium-22, Quaternium-24, Quaternium-26, Quaternium-27, Quaternium-30, Quaternium-33, Quaternium-53, Quaternium-60, Quaternium-61, Quaternium-72, Quaternium-78, Quaternium-80, Quaternium-81, Quaternium-82, Quaternium-83 and/or Quaternium-84.

Further suitable cationic polymers are for example—cationic alkyl polyglycosides, —cationized honey, for example the commercial product Honeyquat® 50, —vinylpyrrolidone —vinylimidazolium methochloride copolymers such as are marketed with the names Luviquat® FC 370, FC 550, FC 905 and HM 552, —quaternised polyvinyl alcohol, —and the polymers with quaternary nitrogen atoms in the main polymer chain known by the names Polyquaternium-2, Polyquaternium-17, Polyquaternium-18 and Polyquaternium-27, —vinylpyrrolidone vinyl caprolactam acrylate terpolymers with acrylic acid esters and acrylic acid amides as the third monomer building block such as are available commercially with the name Aquaflex® SF 40 for example.

In a further preferred embodiment, the hair treatment products as contemplated herein contain from about 0.01 to about 0.5% by weight of at least one cationic tenside c) and from about 0.01 to about 0.5% by weight of at least one cationic polymer d)—relative to the total weight of the water phase (I)—in the water phase (I).

Protein hydrolysates (PH) of both plant and animal, marine or synthetic origin are understood to be PH that are suitable for use in the hair treatment products as contemplated herein.

Preferred animal protein hydrolysates are for example elastin, collagen, keratin, silk and milk protein hydrolysates, which may also be present in the form of salts. Such products are marketed for example under the trademarks Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex) and Kerasol® (Croda).

Preferred plant protein hydrolysates are for example soya, almond, pea, potato and wheat protein hydrolysates. Such products are available for example under the trademarks Gluadin® (Cognis), DiaMin® (Diamalt), Lexein® (Inolex), Hydrosoy® (Croda), Hydrolupin® (Croda), Hydrosesame® (Croda), Hydrotritium® (Croda) and Crotein® (Croda).

The suitable protein hydrolysates of marine origin include for example collagen hydrolysates from fish or seaweed and protein hydrolysates from shells and pearls. Examples of suitable pearl hydrolysates as contemplated herein are the commercial products Pearl Protein Extract BG® or Crodarom® Pearl.

Suitable cationized protein hydrolysates may also be of animal, plant or marine origin.

The quaternisation of the protein hydrolysates or the amino acids may be carried out with quaternary ammonium salts such as N,N-Dimethyl-N-(n-Alkyl)-N-(2-hydroxy-3-chloro-n-propyl)-ammonium halides for example. In addition, the cationic protein hydrolysates may also be derivatised further.

The products known by the following INCI names may be cited as typical examples of particularly suitable cationic protein hydrolysates and/or the derivatives thereof: Cocodimonium Hydroxypropyl Hydrolyzed Collagen. Cocodimonium Hydroxypropyl Hydrolyzed Casein, Cocodimonium Hydroxypropyl Hydrolyzed Collagen, Cocodimonium Hydroxypropyl Hydrolyzed Hair Keratin, Cocodimonium Hydroxypropyl Hydrolyzed Keratin, Cocodimonium Hydroxypropyl Hydrolyzed Rice Protein, Cocodimonium Hydroxypropyl Hydrolyzed Silk, Cocodimonium Hydroxypropyl Hydrolyzed Soy Protein, Cocodimonium Hydroxypropyl Hydrolyzed Wheat Protein, Cocodimonium Hydroxypropyl Silk Amino Acids, Hydroxypropyl Arginine Lauryl/Myristyl Ether HCl, Hydroxypropyltrimonium Gelatin, Hydroxypropyltrimonium Hydrolyzed Casein. Hydroxypropyltrimonium Hydrolyzed Collagen, Hydroxy propyltrimonium Hydrolyzed Conchiolin Protein. Hydroxypropyltrimonium Hydrolyzed keratin, Hydroxypropyltrimonium Hydrolyzed Rice Bran Protein, Hydroxypropyltrimonium Hydrolyzed Silk, Hydroxypropyltrimonium Hydrolyzed Soy Protein, Hydroxypropyl Hydrolyzed Vegetable Protein, Hydroxypropyltrimonium Hydrolyzed Wheat Protein, Hydroxypropyltrimonium Hydrolyzed Wheat Protein/Siloxysilicate, Laurdimonium Hydroxypropyl Hydrolyzed Soy Protein, Laurdimonium Hydroxypropyl Hydrolyzed Wheat Protein, Laurdimonium Hydroxypropyl Hydrolyzed Wheat Protein/Siloxysilicate, Lauryldimonium Hydroxypropyl Hydrolyzed Casein, Lauyldimonium Hydroxypropyl Hydrolyzed Collagen, Lauryldimonium Hydroxypropyl Hydrolyzed Keratin, Lauryldimonium Hydroxypropyl Hydrolyzed Silk, Lauryldimonium Hydroxypropyl Hydrolyzed Soy Protein. Steardimonium Hydroxypropyl Hydrolyzed Casein, Steardimonium Hydroxypropyl Hydrolyzed Collagen, Steardimonium Hydroxypropyl Hydrolyzed Keratin, Steardimonium Hydroxypropyl Hydrolyzed Rice Protein, Steardimonium Hydroxypropyl Hydrolyzed Silk, Steardimonium Hydroxypropyl Hydrolyzed Soy Protein, Steardimonium Hydroxypropyl Hydrolyzed Vegetable Protein, Steardimonium Hydroxypropyl Hydrolyzed Wheat Protein, Steartrimonium Hydroxyethyl Hydrolyzed Collagen. Quaternium-76 Hydrolyzed Collagen, Quaternium-79 Hydrolyzed Collagen, Quaternium-79 Hydrolyzed Keratin, Quaternium-79 Hydrolyzed Milk Protein, Quaternium-79 Hydrolyzed Silk, Quaternium-79 Hydrolyzed Soy Protein, and Quaternium-79 Hydrolyzed Wheat Protein.

Particularly preferred for use in the hair treatment product as contemplated herein are keratin, silk, milk protein, wheat and/or soya protein hydrolysates, which may be quaternised. Particularly preferred are keratin and/or wheat and/or silk protein hydrolysates, which may be quaternised.

The—optionally quaternised—protein hydrolysates may be used both individually and as a mixture in the hair treatment products as contemplated herein.

For some embodiments of the present disclosure, it may be advantageous if the hair treatment products contain at least one protein hydrolysate of animal or plant origin and at least one quaternised protein hydrolysate of animal or plant origin.

The protein hydrolysates and/or quaternised protein hydrolysates may be contained in the water phase (I) of the hair treatment products as contemplated herein—preferably in quantities from about 0.001 to about 5% by weight, preferably from about 0.0025 to about 2.5% by weight and particularly in quantities from about 0.005 to about 1% by weight relative to the total weight of the water phase (I).

Vitamins, provitamins and vitamin precursors as well as derivatives thereof from the groups A, B, C, E, F and H are understood to be vitamins suitable for use in the hair treatment products as contemplated herein.

The group of substances classified as vitamin A includes retinol (vitamin A₁) and 3,4-didehydroretinal (Vitamin A₂). β-carotene is the provitamin of retinol. Vitamin A acid and its esters, vitamin A aldehyde and vitamin A alcohol and its esters such as the palmitate and the acetate for example lend themselves for use as vitamin A components. The hair treatment products may contain the vitamin A component preferably in quantities from about 0.005-1% by weight relative to the weight of the total product.

The vitamin B group or the vitamin B complex includes among others
Vitamin B₁ (thiamine)
Vitamin B₂ (riboflavin)
Vitamin B₃. This designation often includes the compounds nicotinic acid and nicotinamide (niacinamide). Preferred is nicotinamide, which may preferably be contained in the hair treatment products in quantities from about 0.005 to about 1% by weight, relative to the total product
Vitamin B₅ (pantothenic acid, panthenol and pantolactone).

As part of this group, panthenol and/or pantolactone are used for preference. Usable derivatives of panthenol are particularly the esters and ethers of panthenol and cationically derivatised panthenols. Individual representatives are for example panthenol triacetate, panthenol monoethyl ether and its monoacetate as well as cationic panthenol derivatives. The cited compounds of vitamin B₅ type may be contained in the hair treatment products preferably in quantities from about 0.05-5% by weight, relative to the total product. Quantities from about 0.1-2% by weight are particularly preferred
Vitamin B₆ (pyridoxine as well as pyridoxamine and pyridoxal)
Vitamin B₇—see vitamin H.

Vitamin C (ascorbic acid). Vitamin C may be used in the hair treatment products preferably in quantities from about 0.01 to about 3% by weight, relative to the total product. Its use in the form of the palmitic acid ester, the glucosides or phosphates may be preferred. Its use in combination with tocopherols may also be preferred.

Vitamin E (tocopherols, particularly α-tocopherol). Tocopherol and its derivatives, which particularly include the esters such as the acetate, the nicotinate, the phosphate and the succinate, may be contained in the hair treatment products preferably in quantities from about 0.005-1% by weight relative to the total product.

Vitamin F. The term “vitamin F” is usually understood to refer to essential fatty acids, particularly linoleic acid, linolenic acid and arachidonic acid.

Vitamin H. Vitamin H refers to the compound (3aS,4S,6aR)-2-oxohexahydrothienol[3,4-d]imidazole-4-valeric acid but has now largely been superseded by the common name biotin. Biotin may be contained in the hair treatment products preferably in quantities from about 0.0001 to about 1.0% by weight, particularly in quantities from about 0.001 to about 0.01% by weight.

The hair treatment products as contemplated herein may particularly preferably contain vitamins, provitamins and vitamin precursors from the groups A, B, E and H. Panthenol, pantolactone, pyridoxine and derivatives thereof as well as nicotinic acid and biotin are particularly preferred and may be added to the hair conditioning products as contemplated herein both alone and in combination in the aforementioned quantities.

Suitable active agents for pH regulation which are suitable for use in the hair treatment products as contemplated herein are preferably understood to be acids, particularly such as acetic acid, citric acid, malic acid, maleic acid, formic acid, sulfamic acid, phosphoric acid, phosphonic acids, D-lactic acid, L-lactic acid, D/L-lactic acid, oxalic acid, or from the group of alkalis particularly such as caustic soda, advantageously in quantities from about 0.01-10% by weight, preferably from about 0.01-5% by weight and particularly from about 0.01 to about 2% by weight (relative to the total weight of the hair treatment product).

Active agents for pH regulation which are able to guarantee positive properties on the hair and/or scalp besides their primary function of adjusting the pH are preferred for use in the hair treatment products as contemplated herein. In this context, lactic acid is particularly noteworthy, because on the scalp it can help to support regulation of skin moisture and if applicable to protect the colour of dyed hair.

It was found that for particularly good stability of the two phases (I) and (II) of the hair treatment products as contemplated herein it is advantageous if they only contain certain tensides and/or emulsifiers in certain quantities.

In this context, the term “stability” is understood to mean that after thorough mixing the two phases separate quickly again and form a well-defined vertical phase boundary on which ideally no turbidities occur for a long period and in spite of temperature variations.

Consequently, tensides and/or emulsifiers which promote the emulsification and/or suspension of the oil phase in the water phase or vice versa excessively are not suitable for use in the compositions as contemplated herein.

Foaming when the hair treatment product as contemplated herein is shaken immediately before application is also undesirable, so tensides/emulsifiers that tend to form excessive foam are also excluded.

In a preferred embodiment, the hair treatment products as contemplated herein contain not more than about 1% by weight, more preferably not more than about 0.8% by weight, particularly preferably not more than about 0.6% by weight and particularly not more than about 0.5% by weight of anionic, amphoteric, zwitterionic and/or non-ionic tensides and/or emulsifiers (relative to the total weight of the hair treatment products).

Within this embodiment, it is particularly preferable if the hair treatment products as contemplated herein do not contain any anionic, amphoteric, zwitterionic and/or non-ionic tensides and/or emulsifiers other than the silicones b-i) described earlier in this application (which may be silicone-emulsifiers).

It was further observed that the presence of various oils and fatty substances makes the separation of the two phases (I) and (II) more difficult after they have been homogenised.

Therefore, hair treatment products as contemplated herein which contain not more than about 0.5% by weight, particularly preferably not more than about 0.3% by weight and in particular not more than about 0.1% by weight of further oils and/or fatty substances (relative to the oil phase (II)) in their oil phase (II) besides the previously described essential and optional contents are particularly favourable.

The hair treatment products as contemplated herein ideally contain no other oil and/or fatty substances, particularly no fatty alcohols other than the components of the oil phase (II) described earlier in this application.

In a further preferred embodiment, hair treatment products as contemplated herein are substantially free from fatty alcohols.

The term “substantially free from” is understood to mean that the hair treatment products as contemplated herein contain not more than about 0.05% by weight, particularly preferably not more than about 0.01% by weight and in particular 0% by weight fatty alcohols relative to their total weight.

In this context, perfume oils are not to be counted with the previously identified oily and fatty substances. These may be used in the oil phase preferably in a quantity of up to about 5% by weight, more preferably about 4% by weight, particularly preferably about 3% by weight and in particular about 2% by weight (relative to the total weight of the oil phase).

The suitable oily and/or fatty substances which may optionally be added to the oil phase (II) of the hair treatment products as contemplated herein preferably include plant oils.

Within the scope of the present disclosure, suitable plant oils are understood to include amaranth seed oil, apricot kernel oil, argan oil, avocado oil, babassu oil, cottonseed oil, borage seed oil, camelina oil, thistle oil, peanut oil, pomegranate seed oil, grapefruit seed oil, hemp oil, rosehip kernel oil, hazelnut oil, elderberry seed oil, currant seed oil, jojoba oil, cocoa butter, linseed oil, macadamia nut oil, maize germ oil, almond oil, marula oil, evening primrose oil, olive oil, palm oil, peach kernel oil, rape oil, rice oil, sea buckthorn flesh oil, sea buckthom kernel oil, sesame oil, shea butter, soya oil, sunflower oil, grapeseed oil, walnut oil or wild rose oil.

Particularly preferred are avocado oil, apricot kernel oil, argan oil, rosehip kernel oil, jojoba oil, almond oil, olive oil, peach kernel oil, sunflower oil and grape seed oil.

In addition, the hair treatment products as contemplated herein may also contain additional active, auxiliary and additive substances, such as for example
structuring substances such as maleic acid
dyes for colouring the product
anti-dandruff agents such as piroctone olamine, zinc omadine and climbazole
cholesterol
complexing agents such as EDTA, NTA, β-alanine diacetic acid and phosphonic acids
pigments
antioxidants and/or UV filters
amphoteric, anionic and/or anionic polymers
plant extracts
penetrating agents and/or swelling agents such as urea, hydantoin and/or derivatives thereof.

The hair conditioning products as contemplated herein preferably have a pH value in the range from about 2 to about 7, preferably from about 2.5 to about 6 and particularly from about 3 to about 5.

Besides an aesthetic appearance, the two-phase hair treatment products as contemplated herein also have the advantage of being easy to manage and simple to manufacture.

The special combination of active ingredients in the hair treatment products guarantees fast mixing of the two phases before their application and a rapid separation of the two phases after application. The hydrophilic phase is based mainly on water, which favours the consumers' needs.

The hair treatment products as contemplated herein are particularly well suited to hair care and lend the hair improved styling properties and a better feel.

The hair treatment products as contemplated herein are sprayable and are suitable for application from a pump dispenser. Accordingly, they are easy to handle and effective in action, because the fine spray mist which exits the pump dispenser reaches all regions of the hair.

A second object of the present disclosure is a method for conditioning hair in which after vigorous shaking a hair treatment product as contemplated herein is applied (preferably sprayed) to the wet or dry hair and is left on the hair until the next time the hair is washed.

A third object of the present disclosure is the use of the hair treatment product as contemplated herein to improve
the feel, particularly of dry hair
the styling properties of hair
the smoothness and softness of hair (non-frizz).

A fourth object of the present disclosure is the use of one or more linear C₉-C₁₅ alkane(s) in cosmetic two-phase systems with two separate, visually discernible continuous phases with a common vertical phase boundary, to improve the speed of separation and to improve the complete separation of the phases after they have been mixed.

The notes regarding the products as contemplated herein also apply for the method as contemplated herein and the uses as contemplated herein.

The following examples are intended to describe the objects of the present disclosure in greater detail without thereby limiting them.

### Examples

The following two-phase hair treatment products as contemplated herein were produced according to the following method (quantities indicated refer to [% by weight]): A—preferably transparent—receptacle was filled with water, optionally in mixture with further auxiliary and active agents, together with linear C₉-C₁₅ alkanes a), optionally in mixture with further auxiliary and active agents.

The fraction by weight of the water was preferably equal to 70 to 98% by weight, and the fraction by weight of the oil phase was preferably equal to 2 to 30% by weight of the total weight of the hair treatment product.

In the resting state, the two-phase hair treatment products had two separate, visually discernible continuous phases with a common vertical phase boundary.

| | 1 | 2 | 3 | 4 |
|---|---|---|---|---|
| Oil phase | | | | |
| Linear C₉-C₁₅ alkane | ≥10 | ≥20 | ≥25 | ≥30 |
| Water phase | | | | |
| Water | ≥75 | ≥80 | ≥85 | ≥90 |
| | 5 | 6 | 7 | 8 |
| Oil phase | | | | |
| Mixture containing at least 85% by weight | ≥10 | ≥20 | ≥25 | ≥30 |
| of at least two alkanes selected from n- | | | | |
| nonane, | | | | |
| n-undecane, n-tridecane, n-tetradecane and | | | | |
| n-pentadecane | | | | |
| Water phase | | | | |
| Water | ≥75 | ≥80 | ≥85 | ≥90 |
| | 9 | 10 | 11 | 12 |
| Oil phase | | | | |
| Mixture containing at least 85% by weight | ≥10 | ≥20 | ≥25 | ≥30 |
| of | | | | |
| n-undecane and n-tridecane* | | | | |
| Water phase | | | | |
| Water | ≥75 | ≥80 | ≥85 | ≥90 |
| | 13 | 14 | 15 | 16 |
| Oil phase | | | | |
| Linear C₉-C₁₅ alkane | ≥10 | ≥20 | ≥25 | ≥30 |
| Volatile silicone b) | ≥10 | ≥20 | ≥25 | ≥30 |
| Water phase | | | | |
| Water | ≥75 | ≥80 | ≥85 | ≥90 |
| | 17 | 18 | 19 | 20 |
| Oil phase | | | | |
| Mixture containing at least 85% by weight | ≥10 | ≥20 | ≥25 | ≥30 |
| of at least two alkanes selected from n- | | | | |
| nonane, n-undecane, n-tridecane, n- | | | | |
| tetradecane and | | | | |
| n-pentadecane | | | | |
| Volatile silicone b) | ≥10 | ≥20 | ≥25 | ≥30 |
| Water phase | | | | |
| Water | ≥75 | ≥80 | ≥85 | ≥90 |
| | 21 | 22 | 23 | 24 |
| Oil phase | | | | |
| Mixture containing at least 85% by weight | ≥10 | ≥20 | ≥25 | ≥30 |
| of | | | | |
| n-undecane and n-tridecane* | | | | |
| Volatile silicone b) | ≥10 | ≥20 | ≥25 | ≥30 |
| Water phase | | | | |
| Water | ≥75 | ≥80 | ≥85 | ≥90 |
| | 25 | 26 | 27 | 28 |
| Oil phase | | | | |
| Linear C₉-C₁₅ alkane | ≥10 | ≥20 | ≥25 | ≥30 |
| Hexamethyldisiloxane, | ≥10 | ≥20 | ≥25 | ≥30 |
| octamethyltrisiloxane, | | | | |
| decamethyltetrasiloxane, | | | | |
| hexamethylcyclotrisiloxane (D3), | | | | |
| octamethylcyclotetrasiloxane (D4), | | | | |
| decamethylcyclopentasiloxane (D5), | | | | |
| dodecamethylcyclohexasiloxane (D6) | | | | |
| and/or Phenyl Trimethicone | | | | |
| Water phase | | | | |
| Water | ≥75 | ≥80 | ≥85 | ≥90 |
| | 29 | 30 | 31 | 32 |
| Oil phase | | | | |
| Mixture containing at least 85% by weight | ≥10 | ≥20 | ≥25 | ≥30 |
| of | | | | |
| n-undecane and n-tridecane* | | | | |
| Hexamethyldisiloxane, | ≥10 | ≥20 | ≥25 | ≥30 |
| octamethyltrisiloxane, | | | | |
| decamethyltetrasiloxane, | | | | |
| hexamethylcyclotrisiloxane (D3), | | | | |
| octamethylcyclotetrasiloxane (D4), | | | | |
| decamethylcyclopentasiloxane (D5), | | | | |
| dodecamethylcyclohexasiloxane (D6) | | | | |
| and/or Phenyl Trimethicone | | | | |
| Water phase | | | | |
| Water | ≥75 | ≥80 | ≥85 | ≥90 |
| | 33 | 34 | 35 | 36 |
| Oil phase | | | | |
| Linear C₉-C₁₅ alkane | 10-60 | 12-58 | 15-55 | 20-50 |
| Hexamethyldisiloxane, | 20-80 | 22-75 | 25-70 | 30-60 |
| octamethyltrisiloxane, | | | | |
| decamethyltetrasiloxane, | | | | |
| hexamethylcyclotrisiloxane (D3), | | | | |
| octamethylcyclotetrasiloxane (D4), | | | | |
| decamethylcyclopentasiloxane (D5), | | | | |
| dodecamethylcyclohexasiloxane (D6) | | | | |
| and/or Phenyl Trimethicone | | | | |
| PEG/PPG(x/y) dimethicone and/or R- | 0-30 | 1-27 | 3-25 | 5-20 |
| PEG/PPG(x/y) dimethicone and/or | | | | |
| Dimethiconol | | | | |
| Water phase | | | | |
| Water | ≥75 | ≥80 | ≥85 | ≥90 |
| | 37 | 38 | 39 | 40 |
| Oil phase | | | | |
| Mixture containing at least 85% by weight | 10-60 | 12-58 | 15-55 | 20-50 |
| n-undecane and n-tridecane* | | | | |
| Cyclomethicone and/or Phenyl | 20-80 | 22-75 | 25-70 | 30-60 |
| Trimethicone | | | | |
| Cetyl PEG/PPG-10/1 Dimethicone | 0-30 | 1-27 | 3-25 | 5-20 |
| Water phase | | | | |
| Water | ≥75 | ≥80 | ≥85 | ≥90 |
| | 41 | 42 | 43 | 44 |
| Oil phase | | | | |
| Mixture containing at least 85% by weight | ≥10 | ≥20 | ≥25 | ≥30 |
| n-undecane and n-tridecane* | | | | |
| Water phase | | | | |
| Water and optionally agents for pH | ≥75 | ≥80 | ≥85 | ≥90 |
| regulation, preservatives, dyes | | | | |
| | 45 | 46 | 47 | 48 |
| Oil phase | | | | |
| Mixture containing at least 85% by weight | ≥10 | ≥20 | ≥25 | ≥30 |
| n-undecane and n-tridecane* | | | | |
| Water phase | | | | |
| Cationic tensides and/or | 0.01-0.5 | 0.05-0.4 | 0.075-0.3 | 0.1-0.2 |
| cationic polymers and/or | 0.01-0.5 | 0.05-0.5 | 0.1-0.5 | 0.2-0.5 |
| protein hydrolysates and/or | 0.001-5 | 0.002-4 | 0.0025-2.5 | 0.005-1 |
| vitamins | 0.001-1 | 0.002-1 | 0.003-1 | 0.005-1 |
| Water and optionally agents for pH | ≥75 | ≥80 | ≥85 | ≥90 |
| regulation, preservatives, dyes | | | | |
| | 49 | 50 | 51 | 52 |
| Oil phase | | | | |
| Mixture containing at least 85% by weight | ≥10 | ≥20 | ≥25 | ≥30 |
| n-undecane and n-tridecane* | | | | |
| Water phase | | | | |
| Cationic tensides and/or | 0.01-0.5 | 0.05-0.4 | 0.075-0.3 | 0.1-0.2 |
| cationic polymers and/or | 0.01-0.5 | 0.05-0.5 | 0.1-0.5 | 0.2-0.5 |
| protein hydrolysates and/or | 0.001-5 | 0.002-4 | 0.0025-2.5 | 0.005-1 |
| vitamins | 0.001-1 | 0.002-1 | 0.003-1 | 0.005-1 |
| Water and optionally agents for pH | to 100 | to 100 | to 100 | to 100 |
| regulation, preservatives, dyes | | | | |
| | 53 | 54 | 55 | 56 |
| Oil phase | | | | |
| Mixture containing at least 85% by weight | ≥10 | ≥20 | ≥25 | ≥30 |
| n-undecane and n-tridecane* | | | | |
| Hexamethyldisiloxane, | ≥10 | ≥20 | ≥25 | ≥30 |
| octamethyltrisiloxane, | | | | |
| decamethyltetrasiloxane, | | | | |
| hexamethylcyclotrisiloxane (D3), | | | | |
| octamethylcyclotetrasiloxane (D4), | | | | |
| decamethylcyclopentasiloxane (D5), | | | | |
| dodecamethylcyclohexasiloxane (D6) | | | | |
| and/or Phenyl Trimethicone | | | | |
| Water phase | | | | |
| Cationic tensides and/or | 0.01-0.5 | 0.05-0.4 | 0.075-0.3 | 0.1-0.2 |
| cationic polymers and/or | 0.01-0.5 | 0.05-0.5 | 0.1-0.5 | 0.2-0.5 |
| protein hydrolysates and/or | 0.001-5 | 0.002-4 | 0.0025-2.5 | 0.005-1 |
| vitamins | 0.001-1 | 0.002-1 | 0.003-1 | 0.005-1 |
| Water and optionally agents for pH | to 100 | to 100 | to 100 | to 100 |
| regulation, preservatives, dyes | | | | |
| | 57 | 58 | 59 | 60 |
| Oil phase | | | | |
| Mixture containing at least 85% by weight | 10-60 | 12-58 | 15-55 | 20-50 |
| n-undecane and n-tridecane* | | | | |
| Cyclomethicone and/or Phenyl | 20-80 | 22-75 | 25-70 | 30-60 |
| Trimethicone | | | | |
| Cetyl PEG/PPG-10/1 Dimethicone | 0-30 | 1-27 | 3-25 | 5-20 |
| Water phase | | | | |
| Cationic tensides and/or | 0.01-0.5 | 0.05-0.4 | 0.075-0.3 | 0.1-0.2 |
| cationic polymers and/or | 0.01-0.5 | 0.05-0.5 | 0.1-0.5 | 0.2-0.5 |
| protein hydrolysates and/or | 0.001-5 | 0.002-4 | 0.0025-2.5 | 0.005-1 |
| vitamins | 0.001-1 | 0.002-1 | 0.003-1 | 0.005-1 |
| Water and optionally agents for pH | to 100 | to 100 | to 100 | to 100 |
| regulation, preservatives, dyes | | | | |

*Cetiol Ultimate ® (INCI name: Undecane, Tridecane); BASF

The following composition (I) as contemplated herein was compared with compositions from the related art (II) and (III). The indicated quantities refer to % by weight (of the total composition):

| | (I) | (II) | (III) |
|---|---|---|---|
| Oil phase | | | |
| Mixture containing at least 85% by weight | 3 | | |
| n-undecane and n-tridecane* | | | |
| Cyclopentasiloxane | | 3 | 3 |
| Diheptyl Succinate and Capryloyl | | | 4.5 |
| Glycerin/Sebacic Acid Copolymer*** | | | |
| Phenyl Trimethicone | 1 | 1 | 1 |
| Cyclomethicone, Dimethiconol** | 4.5 | 4.5 | |
| Cetyl PEG/PPG-10/1 Dimethicone | 0.5 | 0.5 | 0.5 |
| Water phase | | | |
| Cetrimonium Chloride | 0.15 | 0.15 | 0.15 |
| Polyquaternium-16 | 0.4 | 0.4 | 0.4 |
| Hydrolyzed Keratin | 0.005 | 0.005 | 0.005 |
| Panthenol | 0.2 | 0.2 | 0.2 |
| Water, agents for pH-regulation (pH 4), | to 100 | to 100 | to 100 |
| Preservatives, dyes | | | |

*Cetiol Ultimate ® (INCI name: Undecane, Tridecane); BASF
**DC 1501 ® (INCI name: Cyclomethicone, Dimethiconol); Dow Corning
***Lexfeel N350 ® (INCI name: Diheptyl Succinate and Capryloyl Glycerin/Sebacic Acid Copolymer); Inolex

9% by weight of the respective oil phases and 93% by weight of the respective water phases (in examples (I) to (III) were placed together in a transparent container and left to stand for 48 hours.

In the resting state, the three compositions (I) to (III) had the following appearance:
Composition (I) had a highly transparent water phase and a milky-creamy oil phase. A sharp phase boundary was discernible between the two phases;
The water phase in composition (II) was slightly cloudy and the oil phase displayed slight foaming;
The oil phase in composition (III) was very turbid, the oil phase was foamy and a distinct phase boundary between the two phases was not discernible.

Subsequently, all three compositions were shaken vigorously until the two phases were completely homogenised, after which they were allowed to stand for 40 minutes. After 40 minutes, compositions (I) to (III) had the following appearance:
The water phase of composition (I) was slightly cloudy, but a distinct phase boundary was already discernible between the two phases;
The water phase of composition (II) was very cloudy, and the oil phase was very foamy (bubble formation on the surface);
The water phase of composition (III) was extremely cloudy and the phase boundary was very poorly defined.

In a blind test, the three compositions were also evaluated by test subjects in respect of the care properties
feel (smoothness, softness, silkiness) and
“anti-frizz” effect.

The evaluation concluded that the compositions (I) as contemplated herein received significantly better evaluations than the comparison compositions (II) and (III) in both cases.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A hair treatment product comprising a two-phase system with two separate, visually discernible continuous phases having a common vertical phase boundary, wherein the two phases comprise:
a water phase (I) and an oil phase (II),
wherein the oil phase (II) comprises at least about 10% by weight of at least one linear C₉-C₁₅ alkane a) relative to a total weight of the oil phase (II); and
wherein the water phase (I) comprises at least about 75% by weight water relative to a total weight of the water phase (I).

2. The hair treatment product according to claim 1, wherein the oil phase (II) comprises at least about 15% by weight of the at least one volatile linear C₉-C₁₅ alkane a) relative to the total weight of the oil phase (II).

3. The hair treatment product according to claim 1, wherein the oil phase (II) comprises a mixture of volatile linear saturated C₉-C₁₅ alkanes a), wherein the mixture comprises at least about 85% by weight of at least two alkanes selected from n-nonane, n-undecane, n-tridecane, n-tetradecane and n-pentadecane, based on a total weight of the mixture of volatile linear saturated C₉-C₁₅ alkanes a).

4. The hair treatment product according to claim 1, wherein:
the water phase (I) comprises from about 70 to about 98% by weight of a total weight of the hair treatment product; and
the oil phase (II) comprises from about 2 to about 30% by weight of the total weight of the hair treatment product.

5. The hair treatment product according to claim 1, additionally comprising at least one silicone b) in the oil phase (II).

6. The hair treatment product according to claim 1, comprising from about 0.01 to about 0.5% by weight of at least one cationic tenside c) in the water phase (I) relative to the total weight of the water phase (I).

7. The hair treatment product according to claim 1, comprising from about 0.01 to about 2.5% by weight of at least one cationic polymer d) in the water phase (I) relative to the total weight of the water phase (I).

8. The hair treatment product according to claim 1, comprising at least about 80% by weight water in the water phase (I) relative to the total weight of the water phase (I).

9. The hair treatment product according to claim 1, comprising not more than about 1% by weight anionic, amphoteric, zwitterionic and/or non-ionic tensides and/or emulsifiers relative to a total weight of the hair treatment product.

10. The hair treatment product according to claim 1, wherein the hair treatment product is substantially free from fatty alcohols.

11. The hair treatment product according to claim 1, wherein the water phase (I) and the oil phase (II) each have a viscosity not greater than about 1000 mPas, measured at about 20° C. with a Brookfield DV-II viscometer, spindle 2 at about 20 rpm.

12. The hair treatment product according to claim 1, wherein at least one of the two phases (I) or (II) is transparent.

13. A method for conditioning hair, comprising:
shaking vigorously a hair treatment product according to claim 1; applying the hair treatment product onto the wet or dry hair; and leaving the hair treatment product on in the hair until the next time the hair is washed.

14. The method of claim 13 wherein: leaving the hair treatment product in the hair comprises improving one or more of:
the feel, particularly of dry hair;
the styling properties of hair; and
the smoothness and softness of hair (anti-frizz).

15. The use of one or more linear C₉-C₁₅ alkane(s) in a cosmetic two-phase system with two separate, visually discernible continuous phases having a common vertical phase boundary, wherein the use comprises: improving the speed of separation of the cosmetic two-phase system, and providing complete separation of the phases of the cosmetic two-phase system m after they have been mixed thoroughly.

16. The hair treatment product according to claim 5, wherein the silicon b) has a boiling point of from about 60 to about 260° C.

17. The hair treatment product according to 1, comprising not more than about 0.5% by weight of an anionic, amphoteric, zwitterionic and/or non-ionic tenside and/or emulsifier relative to a total weight of the hair treatment product.

18. The hair treatment product according to claim 1, wherein the water phase (I) and the oil phase (II) each have a viscosity not greater than about 400 mPas, measured at about 20° C. with a Brookfield DV-II viscometer, spindle 2 at about 20 rpm.

19. The hair treatment product according to claim 1, comprising at least about 90% by weight water in the water phase (I), relative to the total weight of the water phase (I).

20. The hair treatment product according to claim 1, wherein the oil phase (II) comprises at least about 30% by weight of the at least one volatile linear C₉-C₁₅ alkane a), relative to the total weight of the oil phase (II).
